(19) United States
(12) Patent Application Publication
(10) Pub. No.: US 2008/0273206 A1
(43) Pub. Date: Nov. 6, 2008

(54) Biomimetic Mineralization Method and System

(75) Inventors: Brian Genge, Columbia, SC (US); Licia Wu, Cayce, SC (US); Roy Wuthier, Columbia, SC (US)

Correspondence Address: DORITY & MANNING, P.A., POST OFFICE BOX 1449, GREENVILLE, SC, 29602-1449, US

(73) Assignee: University of South Carolina, Columbia, SC (US)

(21) Appl. No.: 12/108,041

(22) Filed: Apr. 23, 2008

Related U.S. Application Data
(60) Provisional application No. 60/925,750, filed on Apr. 23, 2007.

(51) Int. Cl. G01N 21/59; G01N 33/48; G01N 33/00

(52) U.S. Cl. 356/441; 702/19; 436/34

(57) ABSTRACT

Disclosed are methods and systems that can be quickly and efficiently utilized to examine the kinetics of a growth and development protocol in a controlled environment, for instance in vivo. Disclosed systems can include a synthetic mineralization complex that can nucleate calcium phosphate mineral deposition in a controlled environment, for instance a controlled environment that can mimic a natural environment in which biomineralization takes place. Also disclosed are non-contact optical methods as may be utilized to examine the kinetics of a developing solid phase. Disclosed systems and methods can be beneficially utilized in high throughput screening in the development of drugs for the treatment and prevention of pathological calcifications such as osteoarthritis and atherosclerosis.

### CROSS REFERENCE TO RELATED APPLICATION

The present application claims filing benefit of U.S. Provisional Patent Application Ser. No. 60/925,750 having a filing date of Apr. 23, 2007, which is incorporated herein in its entirety.

### BACKGROUND

Biomineralization is a physiological process by which living cells or organized tissues become calcified by the precipitation of calcium salts. The organism provides the specialized molecular machinery and matrix that control the nucleation and growth of the mineral. The result is conducive to the formation of discrete and organized calcium precipitates and can also impart hierarchical structural and long range order to the calcifying tissue. Biological calcification is a widespread phenomenon that occurs in bacteria, algae, mussels and vertebrates.

In humans, normal biomineralization occurs during growth and development in a variety of tissues, for example, dental enamel in the formation of teeth, the calcification of growth plate cartilage during the formation of long bones and the healing fracture callus. Pathological calcifications, on the other hand, play a role in diseases such as osteoarthritis, atherosclerosis, kidney stone formation and the degeneration of bioprosthetic heart valves.

Understanding the biomineralization process has been an important goal in biological and medicinal research. For instance, studies of isolated native matrix vesicles (MV) (microstructures involved in the initiation of mineral deposition in bone, cartilage, tendon and a variety of other tissues) have shown that natural mineralization follows a characteristic sigmoid pattern in which there is a lag period before discernible mineral formation begins. Following the lag period is a period of rapid mineral formation, a transient period when the rate obviously declines, and an extended period at a progressively slower rate. In addition, studies have shown that amorphous calcium phosphate (ACP) is of importance during natural mineralization. ACP is a kinetically unstable mineral that forms only when Ca²⁺ and Pi (inorganic phosphate) are both present in high levels. Other components understood to play a role in initiation of the first steps of natural mineral formation include the phospholipid phosphatidylserine (PS) and annexin a5 (AnxA5), a major Ca²⁺-binding protein of MV that has co-dependent affinity for both PS and Ca²⁺.

Unfortunately, existing mineralization models are complicated cell based assays that often require animal testing and/or require the use of radioactive ⁴⁵Ca or ⁸⁵Sr, for example. Accordingly, the capability of examining the effect of active agents on mineralization, including both naturally occurring agents and potential treatment agents, has been both time consuming and expensive. For example, the ability to obtain data with regard to the influence of an agent on the mineralization induction time (T_{I}), the initial rate of mineral formation (RMF_{R}), the maximal amount of mineral formed (AMF_{Max}), and the nucleation potential (NP) (a parameter than defines the ability of nucleators to induce and propagate mineral formation) has been expensive and arduous.

The development of a simple, accurate and robust biomineralization model is needed to gain further insight into the mechanism of mineralization as well as to accelerate the discovery of drugs for the treatment and prevention of pathological calcification diseases such as osteoarthritis.

### SUMMARY

According to one embodiment, disclosed is an in vitro calcium phosphate mineralization method. A method can include, for example, forming a synthetic mineralization complex that can nucleate calcium phosphate mineral deposition in a controlled environment, i.e., an environment in which at least one of the parameters defining the environment (temperature, pressure, sample volume, etc., is under the control of an operator). For purposes of the present disclosure, the term ‘synthetic’ with regard to a compound can generally refer to a compound at least a portion of the formation of which is controlled or directed by non-natural means. For instance, the individual components of a synthetic complex may be naturally derived, and the process for combining those natural components in known stoichiometric amounts to form a complex can be carried out in a controlled environment, e.g., an in vitro environment. As such, the formed complex is considered a synthetic complex as described herein. A synthetic mineralization complex can include amorphous calcium phosphate and a lipid such as, for example, a phospholipid such as phosphatidylserine. According to one embodiment, a synthetic mineralization complex can include additional components such as, for instance, an annexin protein (e.g., annexin A5).

In one embodiment, a synthetic mineralization complex can be formed in a synthetic intracellular phosphate (ICP) buffer, for instance an ICP that mimics the intracellular environment of a growth plate chondrocyte.

A method can further include locating a synthetic mineralization complex in a controlled environment, e.g., an in vitro environment. An in vitro environment can mimic a natural extracellular environment in which biomineralization occurs. For example, an in vitro environment can mimic the extracellular environment of a growth plate chondrocyte or can mimic a cartilage fluid, blood, serum, or the like. Following location of a synthetic mineralization complex in an appropriate environment, the mineralization complex can nucleate deposition of a calcium phosphate mineral phase in the environment. The mineral phase formed can mimic the poorly crystalline hydroxyapatite mineral formed by matrix vesicles and found in bone.

A method can also include monitoring the turbidity of a controlled environment according to an optical analysis technique. For instance, the absorbency data of an environment can be gathered through utilization of spectrophotometer as is known in the art.

According to another embodiment, disclosed is a method for examining the kinetics of formation of a solid phase. A solid phase can be, by way of example, a calcium phosphate mineral solid phase or a biofilm. A method can include forming the solid phase in a controlled environment, monitoring the turbidity of the environment according to a non-radioactive optical analysis technique, gathering the turbidity data over time, and carrying out mathematical analyses of the data to obtain desired kinetic information. For instance, a first derivative analysis can be carried out on the turbidity data to obtain one or more kinetic parameters of the formation process.

According to another embodiment, disclosed are systems that can be utilized in carrying out the disclosed methods. For example, a system can include a controlled environment including a synthetic mineralization complex and can also include an optical device in optical communication with the controlled environment for monitoring the turbidity of the environment. A system can include additional components as well such as, for example, a data analysis component that can receive turbidity data from an optical device and display and/or mathematically manipulate the data to provide information about the environment to a user.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures, in which:

FIG. 1 illustrates experimentally obtained mineralization formation data in conjunction with calculated kinetic data obtained from the experimental data according to methods as described herein;

FIG. 2 illustrates the results of FIG. 1 upon performance of an iterative process to minimize the sum of the square of the error difference between the calculated and the experimental data;

FIG. 3 graphically illustrates kinetic parameters of a mineralization system as described herein;

FIGS. 4A graphically illustrates data obtained during mineralization methods as described herein including a variety of different mineralization nucleators;

FIG. 4B illustrates the effects of the addition of native type II collagen to the mineralization systems of FIG. 4A.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is related to the development and examination of controlled systems during the development of a solid phase therein, and in one preferred embodiment, biomimetic calcification systems. More specifically, disclosed herein are systems that can be quickly and efficiently utilized to examine the kinetics of a solid phase growth and development protocol. For example, disclosed systems and methods can be beneficially utilized for in vitro high throughput screening in the development of drugs for the treatment and prevention of pathological calcifications such as osteoarthritis and atherosclerosis. Moreover, while disclosed systems can be utilized in one preferred embodiment for the development and examination of biomimetic mineralization systems, the present disclosure is not limited to either mineralization systems or biological-based systems.

For example, in another embodiment, disclosed systems can be utilized in examination of the kinetics of development of salt water calcification of a biofilm. Biofilms are complex aggregations of microorganisms marked by the excretion of a protective and adhesive matrix and are thought to be involved in a variety of microbial infections such as dental plaque formation, urinary tract infections and chronic sinusitis.

Disclosed examination methods are based on optical effects, e.g., light scattering, by the nascent components of the system. Beneficially, disclosed systems can monitor developing formations without disturbing the system through use of an optical analysis technique, for example through use of an automated plate reader that measures absorbance of the local environment in which mineralization occurs. Disclosed examination methods can yield precise replicate values that typically agree within less than about 5%. Moreover, analysis of data obtained from disclosed systems can provide detailed information with regard to the effect of one or more active agents on the kinetics of specific portions of an overall growth mechanism.

According to another embodiment, disclosed herein are synthetically prepared mineralization complexes as may be utilized in one application to examine mineralization protocols in a controlled environment. Synthetic complexes have been developed based on the mineralization core of components that have been identified and isolated from native matrix vesicles and can, in one embodiment, be built from purified biological components found in calcified tissues. For instance, synthetic complexes disclosed herein can recapitulate in vitro core complexes utilized for mineral formation in vivo. Through the synthetic reconstitution of core biological components believed to be involved in mineralization in vivo, mineralization can be accurately, precisely and reliably reproduced in vitro. As such, disclosed complexes have as one potential use the screening for and potential discovery of a wide variety of drugs for treatment of prevention of diseases, e.g., hypermineralizaing diseases. For example, disclosed methods and systems can be useful in examination and development of treatment of disease ranging from osteoarthritis to atherosclerosis as well as other ectopic mineralization diseases and soft tissue mineralization.

Mineralizing complexes disclosed herein include amorphous calcium phosphate (ACP) in conjunction with at least one lipid that is capable of forming a complex with calcium ion. In one embodiment, a mineralizing complex can include a phospholipid. For instance, in one preferred embodiment, an acidic phospholipid, such as phosphatidylserine (PS) can be utilized. PS may be preferred in some embodiments as it is known to be present in high quantities is matrix vesicles and has a high affinity for calcium ion. Other lipids as may form a complex with ACP can include other acidic lipids such as the acidic phosphatidic acid, and amphiphilic lipids, such as phosphatidylinositol, sphingomyelin, cholesterol, cardiolipin and the like. In another embodiment, a mixture of lipids can be utilized to form a mineralizing complex.

In order to provide a synthetic complex that can accurately mimic biological mineralization nucleators, it can be beneficial to form the disclosed complexes in a buffer that mimics the intracellular environment in which natural mineralization cores are formed. For instance, in one embodiment, a complex can be formed in a buffer including electrolyte content similar to that observed in growth plate chondrocytes, articular chondrocytes, osteoblasts, odontoblasts, and so forth.

For example, a phosphate buffer as may be utilized in formation of a mineralization complex can include concentrations of potassium ion, sodium ion, magnesium ion, chloride ion, inorganic phosphate, carbonate ion, sulfate ion, and so forth so as to mimic the intracellular environment in which natural mineralization core can be formed. In one embodiment, synthetic mineralization complex can be formed in an intracellular phosphate buffer (ICP) that can include between 0 and about 250 (e.g., about 106.7) mM K⁺, between 0 and about 250 (e.g., about 45.1) mM Na⁺, between 0 and about 10 (e.g., about 1.5) mM Mg²⁺, between 0 and about 250 (e.g., about 115.7) mM Cl⁻, between about 0.1 and about 100 (e.g., about 23.0) mM Pi, between about 0.1 and about 100 (e.g., about 10) mM HCO₃⁻, between 0 and about 10 (e.g., about 1.5) mM SO₄²⁻. In general, a phosphate buffer utilized in formation of disclosed mineralization complexes can also include a preservative, for instance, between 0 and about 5 (e.g., about 3.1) mM of a preservative, such as N₃⁻ or antibiotic/antimicotic agent such as streptomycin penicillin, amphotericin B, and so forth. Accordingly, a phosphate buffer can, in one embodiment have a total molarity of between about 0.1 and about 500 millimolar, for instance, about 153.3 mM. In order to form a mineralization complex as desired, a phosphate buffer should have a pH within a fairly narrow range, for instance, between about 7.0 and about 8.2. In one preferred embodiment, the pH of an ICP buffer can be 7.2

Synthetic mineralization complexes can include compounds in addition to calcium, phosphate, and a lipid. In vivo, it is believed that initial biomineral deposition begins by uniquely arranging key proteins, lipids and ions with atomic level precision. Subsequent mineral growth takes place in the surrounding matrix. Cellular processes and matrix components direct spatial and temporal mineral deposition to create a hierarchical biocomposite. The resulting in vivo structure has increased load strength and durability that is essential for weight-bearing tissues such as bone.

To mimic this process in vitro, components involved in the mineralization process can be purified and quantitatively reconstituted. For instance, in addition to calcium ions and inorganic phosphate, proteins that are known or believed to be involved in mineralization can be included in a complex. By way of example, annexins 5, 2, and 6 are quantitatively major proteins of the matrix vesicle nucleational core that is responsible for mineral formation and can be included as a component in a synthetic mineralization complex as described herein.

Proteins as may be incorporated into a disclosed synthetic mineralization complex can include any suitable proteins including purified natural protein, recombinant protein, and the like. For instance, native human annexin protein (e.g., as may be purified from human placenta according to standard methods as are generally known in the art) may be utilized as well as native proteins obtainable from other species such as poultry annexin (e.g., as can be isolated from chicken cartilage according to known methods), bovine annexin, porcine annexin, and so forth may be utilized. Recombinant proteins, for instance, recombinant annexin proteins are available from a variety of sources (e.g., Bender MedSystems of Burlingame, Calif.; R&D Systems of Minneapolis, Minn.; Genway Biotech, Inc. of San Diego, Calif.; and Aniara Corporation of Mason, Ohio).

In one preferred embodiment, Annexin-A5 (Anx-A5) protein can be incorporated in a mineralization complex. According to this embodiment, a complex can be precipitated, for instance in an ICP, under suitable conditions (examples of which are described further in the Example section, below) to form a quaternary complex between a protein (e.g., Annexin V), a lipid (e.g., a phospholipid), calcium ion and inorganic phosphate.

A synthetic mineralization complex can nucleate formation of a calcium phosphate phase in any suitable environment, and in particular, any suitable controlled environment. In one embodiment, controlled environment can be an in vitro formation environment that can mimic the extracellular environment in which bulk mineralization can be carried out in vivo. For instance, an in vitro formation process nucleated by a synthetic mineralization complex can mimic the kinetics of mineral formation by isolated matrix vesicles—the principle nucleating agent in vivo. A controlled environment can mimic any ionic environment known for calcium phosphate phase formation. For example, an in vitro environment can simulate an in vivo environment of growth plate chondrocytes that form MV. Other natural environments that can be simulated in a calcium phosphate phase formation modeling system as disclosed herein can include other in vivo environments such as blood or serum environments. Such a biomimetic system can be utilized, for example, to mimic mineralization within arteries and/or heart valves.

Disclosed methods are not limited to testing/examination of in vivo biological processes. For instance, a controlled environment can be developed that models natural salt water conditions. Such a system can be developed for examination of a salt water calcium phosphate phase formation process, for instance to test for inhibitors or stimulators of scaling on ships.

In one preferred embodiment, synthetic lymph that mimics the electrolytic composition of cartilage fluid can be utilized to encourage bulk mineralization nucleated from a synthetic mineralization complex as described herein. For example, one suitable synthetic cartilage fluid can include between about 0.1 and about 20 (e.g., about 2) mM Ca²⁺ and between about 0.1 and about 15 (e.g., about 1.42) mM Pi in addition to between 0 and about 250 (e.g., about 104.5) mM Na⁺, between 0 and about 250 (e.g., about 133.5) mM Cl⁻, between 0 and about 250 (e.g., about 63.5 mM) sucrose, between about 0.1 and about 100 (e.g., about 16.5 mM) TES, between 0 and about 100 (e.g., about 12.7) mM K, between 0 and about 100 (e.g., about 5.55) mM glucose, between 0 and about 100 (e.g., about 1.83) mM HCO₃⁻, and between 0 and about 10 (e.g., about 0.57) mM Mg sulfate. The pH of an SCL can generally be between about 7.0 and about 8.0, for instance, about 7.5.

In addition to electrolytes, a mineralization environment can include other materials as may be expected to be found in an in vivo extracellular mineralization environment. For instance, MV mineralization is known to occur in an environment rich in collagen, and in particular type II and type X collagen. The presence of native collagens in the media is believed to further enhance mineral growth. Accordingly, an in vitro mineralization environment can include one or more extracellular matrix proteins, such as type II and/or type X collagen, proteoglycans, hyaluronic acid, osteocalcin, and so forth.

Upon incubation of a mineralization complex in a suitable environment, calcium phosphate mineral deposition can occur. The deposition can, in one embodiment, be a biomimetic process that closely models an in vivo mineralization process. As such, the methods presented describe a robust mineralization model that enables semi-automated systematic study of the effects of numerous factors thought to contribute to mineral formation such as pyrophosphate or the bisphosphonates.

Disclosed methods and systems take advantage of the fact that macromolecular aggregation and particle assembly during a mineralization process can give rise to increased turbidity of the local environment. For instance, as mineralization progresses, synthetic cartilage lymph in which mineralization takes place will exhibit increased turbidity. This can provide a route for monitoring a system via optical processes, for instance through the monitoring of increased light scattering of a system as mineralization progresses.

Utilization of optical analysis techniques in examination of a solid phase formation system can provide non-destructive analysis of a process and can also allow complete sample recovery at any point during a procedure. In addition, examination of the obtained optical data can provide information with regard to kinetics of a mineralization process in real-time without the use of any additional detectable tags or markers, and in particular, without the need for any radioactive materials. Furthermore, the analysis methods and techniques described herein are not limited to examination of biomimetic mineralization processes and can be utilized to monitor and analyze any controlled system that is characterized by changing optical characteristics as a system progresses through a solid phase formation. For instance, disclosed monitoring and analysis processes can be used for monitoring the formation of biofilms in vitro.

Disclosed systems can provide improved understanding of the regulation of mineral formation through improved definition and direct measurement of different phases of a process. Disclosed systems can also provide information with regard to the individual contribution of each phase to an overall mineral-forming process. Models can be reproducible and precise enough to enable accurate measurements of the effect of one or more factors that can effect examined aspects of mineral formation.

According to one embodiment, one or more optical characteristics of a system including, without limitation absorbance, index of refraction, scattering, and so forth can be measured throughout a mineralization process. Any suitable method can be utilized to measure one or more optical characteristics. For instance, a standard optical reader (e.g., a spectrophotometer) as is generally known in the art can be operated in conjunction with a mineralization process and utilized to measure absorbance. In another embodiment measurement of the light scattering of a system over time can be carried out, for instance via utilization of a laser based, particle size analyzer that can provide kinetic data as well as particle size data. According to another embodiment, a refractometer can be utilized to measure the change in refractive index of a system to determine kinetic data about the system as a calcium phosphate solid phase develops.

In one preferred embodiment, a spectrophotometer can be utilized to obtain changing absorbency data from a system. Absorbency data can be obtained at any suitable baseline wavelength. For example, a microplate reader can utilize a baseline wavelength of longer than about 300 nm, and a detector can be utilized to determine the absorbency of the system at periodic intervals. Lower wavelengths may not be preferred, as lower wavelength light could lead to excitation and autofluorescence of proteins contained in a system.

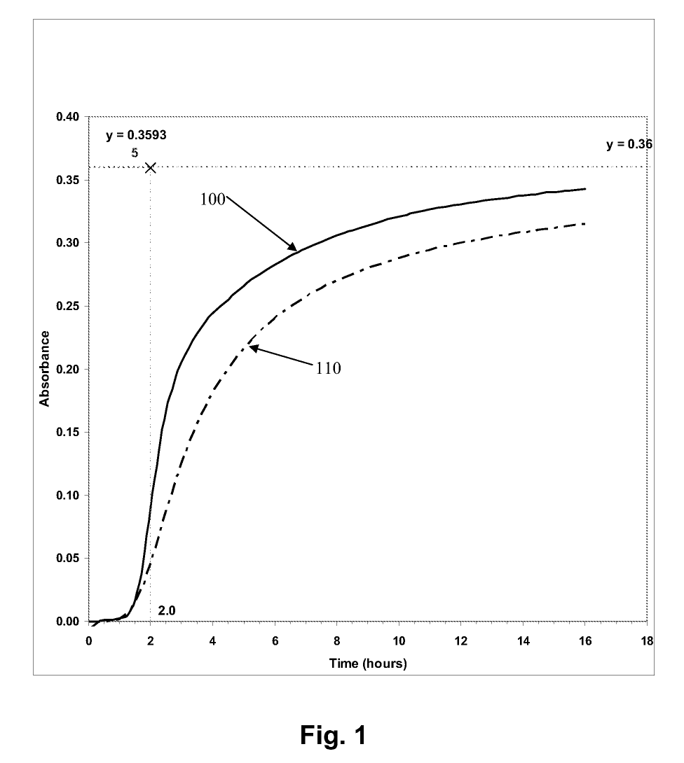

FIG. 1 illustrates a graph of the time and absorbency data obtained in one exemplary process described further in the Example section, below. Specifically, line 100 of FIG. 1 illustrates the best fit of the experimental data. As can be seen, mineral formation nucleated by the synthetic complex follows a sigmoid pattern, similar to mineralization by native matrix vesicles: following a quiescent induction period, rapid formation ensues for a limited time, followed by a distinct decline in rate, which continues to slow, ultimately reaching a maximal asymptotic value.

Quantization of mineral formation through first-derivative analysis of the data can be utilized to precisely obtain several parameters of the system including the induction time, which is the time needed to induce mineral formation in the system (T_{I}); the average rate of mineral formation during the rapid formation period (RMF_{R}); and the nucleation potential (NP=(RMF_{R}/T_{I})×100) of a nucleator (a mineralization complex) used to initiate the process.

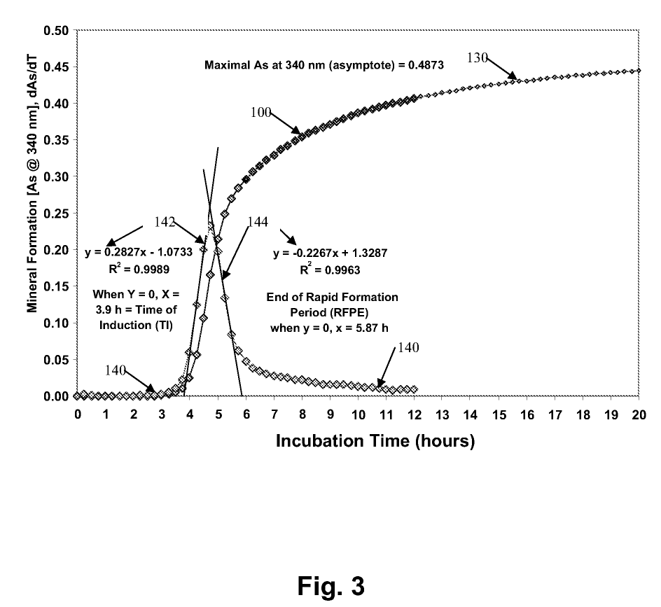

FIG. 3 graphically illustrates the results of the first derivative analysis of absorbency (As)/time (t) data. Specifically, FIG. 3 includes the sigmoidal curve obtained from the experimental data 100 as well as a calculated curve obtained with a 5 parameter approximation fit of the data 130, described below. In addition, FIG. 3 includes the first derivative (dAs/dt) curve 140 with superimposed lines to differentiate the ascending portion of the first derivative 142 and the descending portion 144, as shown. The ascending region 142 extrapolates to the time of induction (T_{I}) at the y-intercept, y=0 (in this example, 3.79 hours). The descending region 144 of the dAs/dt curve extrapolates to the end of the rapid formation period (RFP_{E}) at the y-intercept, y=0 (in this example, 5.87 hours). The rate of rapid mineral formation (RMF_{R}) corresponds to the average slope of the mineral formation curve between T_{I} and RFP_{E} (in this example, 0.137). The RMF_{R} can be calculated by dividing the absorbance at RFP_{E} (0.2903 from FIG. 3) by the total length of the rapid formation period (i.e., RFP_{E}-T_{I}, or 5.87−3.79=2.08 hours). This parameter can in turn be utilized to determine the nucleation potential ((RMF_{R}/T_{I})×100), which is a sensitive measure of the potency of the nucleator of the system (e.g., a synthetic mineralization complex as described above).

Additional data analysis can be utilized to obtain additional parameters of a system. For example, using a 5-parameter logistic curve fitting algorithm, additional kinetic data can be accurately predicted. More specifically, the kinetic profile of mineralization follows a quasi-sigmoidal pattern that can be approximated by the five-parameter logistic fit:

$$y = d + \frac{( a - d )}{{( 1 + {( \frac{x}{c} )}^{b} )}^{g}}$$

wherein:

x=incubation time

y=absorbency

a=baseline absorbency at time=0

b=the slope at the inflection point

c=the time of inflection point

d=the maximal absorbency

g=the asymmetry factor

Given an experimental data set, the equation above can be solved for the other parameters, providing more detailed quantified data of the system. For instance, for a typical analysis of a mineral formation curve, such as that illustrated in FIG. 1, the starting (background) absorbance is determined and subtracted from the raw data points over the entire time course run. An optical device can be in communication (e.g., hard wired, wireless communication, etc.) with a data analysis component, such as a computer, that can be incorporated within an optical device or can be a separate component, as desired. Accordingly, data collected from the system, for instance data with regard to the turbidity of the system, e.g., absorbance values, can be conveyed from the optical device to the data analysis component via device software. The data analysis component can in turn include suitable software that can be utilized to display and/or manipulate the data. For example, a data analysis component can include software capable of solving the above equation. For instance, measured absorbance values can be transferred into a Microsoft® Excel® spreadsheet, which includes imbedded formulas and macros within the program that can automatically generate a plot from the data (see FIG. 1, Exp. Data 100). The software can also contain equation solving capability (the Solver tool) to solve the 5-parameter sigmoidal equation. Utilization of Microsoft® Excel® and related programs is not a requirement of the disclosed subject matter, and any suitable data manipulation method can be utilized. For example, SYSTAT PEAKFIT®, Frontline Systems “SOLVER”, and the like can be utilized to mathematically manipulate experimental data according to known methods.

Using a suitable equation solving software, an operator can make a first approximation to the 5-parameter logistic fit variables by simple estimating values for a, b, c, d, and g of the above equation. As the program updates the values, a plot corresponding to the calculated reaction curve can be formed, for instance overlaid on the curve obtained from the experimental data, as shown in FIG. 1 (Calc. data, 110), and can provide visual feedback with respect to the accuracy of approximating the experimental data.

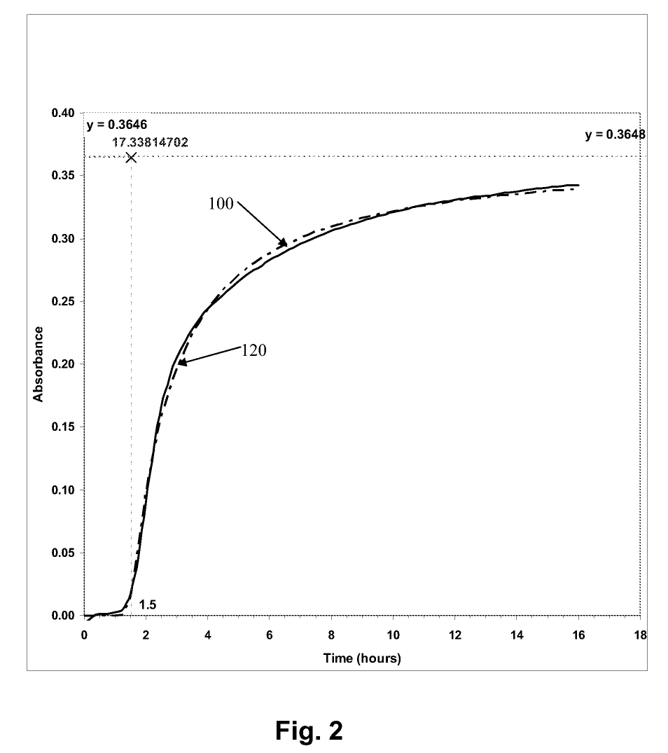

In one embodiment, a data analysis component can include a macro for performing an iterative process to minimize the sum of the square of the error difference between the Experimental and Calculated Data. Execution of the macro can then further adjust the values for a, b, c, d, and g of the above equation, graphical results of which are illustrated in FIG. 2 at 120. The optimized parameters for the logistic fit can be further mathematically manipulated as desired. For example, results can be displayed on a graph and/or relayed into a software program for further processing or otherwise reported and processed as desired. For instance, results can be combined with a weighting factor (default set to “1”) that can be changed to emphasize closer tolerance at specific regions of the mineral pattern.

The calculated solution of the above equation can provide additional information about an experimental system. For instance, the value obtained for ‘d’ in the above equation is the asymptotic absorbency value indicating the maximal absorbency at maximal mineral formation. In comparing different systems for examination of the effect of an agent on the mineralization capabilities of the systems, comparison of the maximal absorbencies of the systems can be utilized to compare total mineralization capability. Alternatively, actual concentration values for total mineral formed can be obtained utilizing the maximal absorbency data through utilization of, for example, formation of a calibration curve. For instance, in an embodiment in which total calcium ion available in the controlled environment in which the calcium phosphate mineral phase is formed is about 2 mM, 1 absorbance unit (AU) has been found to be equivalent to approximately 1% of the available calcium. Thus, a final determination of the parameter ‘d’ as being 0.50 can correspond to mineral formation containing 50% of the available calcium ion, or 1 mM calcium.

Solutions for the other parameters of the above equation likewise can provide information about the modeled system. For example, the solution of the parameter ‘b’ can provide indication of the rate of mineral formation. A solution of the parameter ‘c’ can inform as to the time at which maximum rate of mineral formation is occurring. By way of example, when examining different systems containing different components or under different conditions, through comparison of the values obtained for the parameters of the above equation information can be gathered as to the effects upon a system of one or more components or conditions of the systems. For instance, mineral inhibitor agents could be detected by an increase in ‘c’ or a decrease in ‘b’ from one system to another. Conversely, when screening for agents that stimulate mineral formation, lower values of ‘c’ and/or higher values of ‘b’ and ‘d’ could indicate stimulation of formation.

In vitro methods and systems as disclosed and described herein can be used in one embodiment for high throughput screening and discovery of drugs, factors, small molecules, ligands and other agents that may exert inhibitory or stimulatory action on the biomineralization process. For instance, disclosed methods can be utilized to more precisely monitor the effects of various factors on the induction and support of calcium phosphate mineral formation. The use of an in vitro biomineralization assay can expedite the discovery of drugs for the treatment and prevention of diseases related to incomplete mineralization (i.e. chondrodysplasia) or ectopic mineralization such as osteoarthritis. The methods, techniques and scientific principles disclosed herein also could be useful for monitoring the formation of other solid phase biological structures such as biofilms, the presence of which can scatter an impinging light.

The present disclosure may be better understood with reference to the following Example.

### EXAMPLE

A 4× stock emulsion of phosphatidylserine (PS) was prepared by drying 5 mg PS in chloroform under nitrogen to form a thin film in a test tube. Then, 2 ml of an inorganic phosphate (Pi)-rich intracellular phosphate buffer (ICP buffer) was added. This buffer contained 106.7 mM K⁺, 45.1 mM Na⁺, and 1.5 mM Mg²⁺, 115.7 mM Cl⁻, 23.0 mM Pi, 10 mM HCO₃⁻, 1.5 mM SO₄²⁻, and 3.1 mM N₃⁻ as a preservative; its total molarity=153.3 mM; its pH=7.2.

The tube was sonicated for 2-4 min at 25° C. in a water bath to form a uniform translucent emulsion of small unilamellar vesicles. To make a 400 μl solution of a mineralization complex, a 100 μl sample of the above described 4×PS stock emulsion was diluted with 300 μl of ICP buffer (9.2 μmol Pi in 400 μL), and to this was added drop-wise 7.0 μl of 100 mM CaCl₂ (0.7 μmol) with rapid stirring over a 5-10 min period. Upon addition of Ca²⁺ to the ICP buffer, amorphous calcium phosphate (ACP) instantly forms due to the high Ca²⁺×Pi ion product (40 mM², Ca²⁺/Pi mixing ratio=0.07). During the formative period, nascent ACP combined with the PS liposomes to form an insoluble PS-CPLX, which was harvested by centrifugation for 5 min at ˜15,000×g.

In some runs, PS was omitted and the 100 mM CaCl₂ stock was added drop-wise into the Mg²⁺-containing, Pi-rich ICP buffer with rapid stirring over a 5-10 min period to form ICP-based ACP.

Synthetic mineralization complex including ACP, a phospholipid (PS) and annexin 5 was also formed (PS-CPLX-AnxA5). Native chicken liver AnxA5 was purified and dialyzed against the ICP buffer. Aliquots (200 μL) containing 200 μg of the native annexin isolate were combined with 100 μl of the PS stock solution in ICP, the final volume being adjusted to 400 μl before adding CaCl₂ as above. As a control, the purified native AnxA5 was added to the ICP buffer without PS; CaCl₂ was then added to form the ACP-AnxA5 complex, which was harvested by centrifugation.

Intact, native type II collagen containing intact telopeptides was isolated from chicken sternal and growth plate cartilage. The collagen was dialyzed against synthetic cartilage lymph (SCL) and its level measured by SDS-PAGE.

### Mineralization Assay

Mineral formation was measured by turbidity, i.e. absorbency (As) at 340 nm using a multiwell microplate assay system. Following centrifugation of the mineralization complexes, the pellets were resuspended in 1 ml of SCL by brief sonication to yield uniform suspensions. SCL utilized contained 2 mM Ca²⁺ and 1.42 mM Pi in addition to 104.5 mM Na⁺, 133.5 mM Cl⁻, 63.5 mM sucrose, 16.5 mM TES, 12.7 mM K, 5.55 mM glucose, 1.83 mM HCO₃⁻, 0.57 mM Mg sulfate; the pH of SCL was 7.5. As a control to determine if any of the turbidity was due to coalescence of collagen fibrils, in some studies, Pi was omitted from SCL to prevent mineral formation.

Quadruplicate samples (140 μl) of each were successively distributed into wells of a 96-well half-area Costar microplate. Turbidity measurements were automatically made and recorded at 15 min intervals, after brief (5 sec) cyclonic (600 rpm, 5 mm circular displacement) agitation, for 12-16 h using a Labsystems iEMS Reader MF microplate reader (Needham Heights, Mass.).

The baseline absorbency (As) at 340 nm was established, averaging recorded values during the initial incubation period when no statistical change was observed. This baseline was subtracted from all recorded absorbency values to obtain the apparent level of mineral formation. To enable more accurate measurement of each parameter, these baseline-corrected data were smoothed by calculating a running average of each successive 3 measurements.

As controls to ensure that the absorbency measured at 340 nm was due to mineral formation and not to flocculation or aggregation of added mineralization complexes or collagen, the following mineralization complexes, prepared as described above, were incubated in Pi-free SCL which prevented mineral formation: 1) ACP, 2) ACP+avian liver annexin a5 (ACP-AnxA5), 3) PS-CPLX, 4) PS-CPLX+avian liver annexin a5 (PS-CPLX-AnxA5), 5) type II collagen alone in Pi-free SCL, and 6) PS-CPLX-AnxA5+type II collagen in the Pi-free SCL. These nucleators were incubated for up to 24 h in the Pi-free SCL, monitoring turbidity every 15 min.

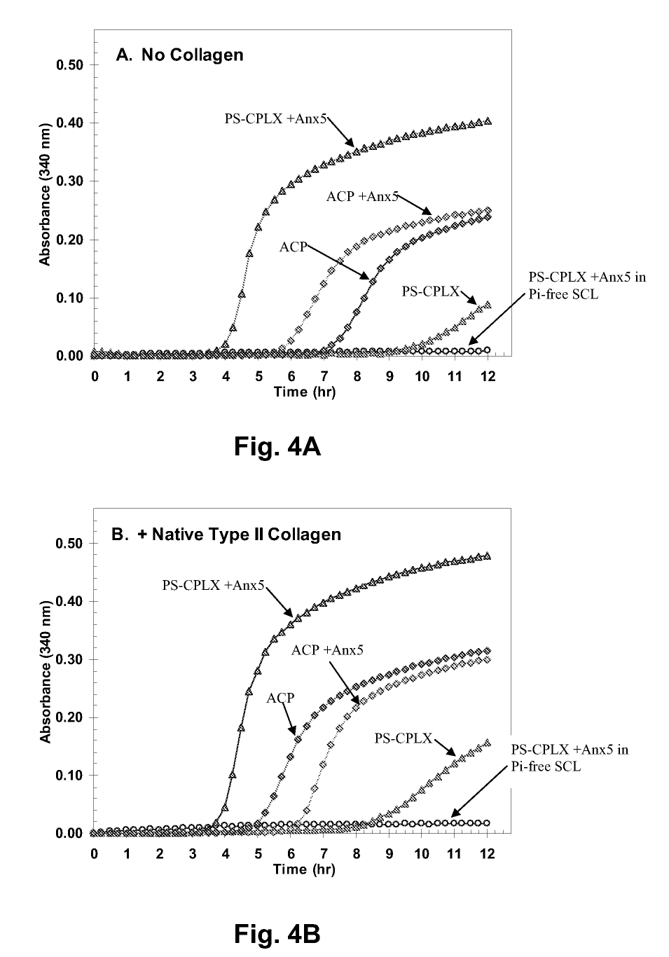

For assay of mineralization without collagen (FIG. 4A and Table 1, below) 60 μL of the various suspensions were added to 1 ml SCL and quadruplicate 140 μL samples of each distributed to the wells of the 96-well Costar microplate. For assay of mineralization with collagen (FIG. 4B and Table 2, below) the setup was the same except that 20 μg of native type II collagen was added to 1 ml SCL.

When each of the nucleators (i.e., the synthetic mineralization complexes) studied were incubated in control Pi-free SCL to prevent mineral formation, plots of absorbency at 340 nm vs. incubation time, revealed minimal increase with time (see, e.g., results for PS-CPLX-AnxA5 in Pi-free SCL on FIGS. 4A and 4B). After 12 h incubation, the maximum absorbencies of the various nucleators incubated in Pi-free SCL ranged from 0.0072±0.0001 to 0.0168±0.0001, values that were only one-twelfth to one-thirtieth of those seen when they were incubated in normal Pi-containing SCL. When incubated even longer, absorbency values increased minimally further. Thus, the increases in absorbency seen when these nucleators were incubated in normal Pi-containing SCL were due to mineral formation, and not to flocculation or aggregation.

Mineral formation induced by nucleators incubated in normal SCL had a characteristic sigmoidal shape (FIGS. 4A and 4B) characterized by: a) an initial lag period in which there was no increase in absorbency at 340 nm (i.e. no mineral formation was evident), b) a transition period when increases in absorbency indicated that mineral formation had begun (the induction time, T_{I}), c) a well-defined period of rapid increase in absorbency (rapid mineral formation), d) a second transition period in which the increase in absorbency slowed, and e) an extended period of progressively slower increase in absorbency, which extrapolated to an asymptote of apparent maximal mineral formation (AMF_{Max}). These parameters were highly reproducible within quadruplicate samples of each treatment, but varied widely between the different nucleators.

The kinetics of mineral formation both with and without the addition of collagen to the system for each nucleator were analyzed as described above. Results are illustrated in FIGS. 4A and 4B, and Tables 1 (no collagen added to the system) and 2 (including collagen), below.

TABLE 1

| | | | | PS- |
|---|---|---|---|---|
| Parameter | ACP | PS-CPLX | ACP + AnxA5 | CPLX + AnxA5 |
| T_{I} (h) | 6.80 ± 0.34 | 9.45 ± 0.47⁻³ | 5.54 ± 0.16⁻² | 3.80 ± 0.01^{-5,-5} |
| RMF_{R} | 0.018 ± 0.003 | 0.040 ± 0.002⁻⁴ | 0.111 ± 0.003⁻³ | 0.297 ± 0.007^{-7,-7} |
| (dAs/DHr) | | | | |
| AMF_{Max} | 0.279 ± 0.019 | 0.182 ± 0.014⁻² | 0.288 ± 0.008 | 0.477 ± 0.005^{-6,-6} |
| NP | 0.96 ± 0.08 | 0.38 ± 0.03⁻⁴ | 1.22 ± 0.05⁻² | 3.64 ± 0.05^{-8,-9} |

TABLE 2

| | | | | PS- |
|---|---|---|---|---|
| Parameter | ACP | PS-CPLX | ACP + AnxA5 | CPLX + AnxA5 |
| T_{I} (h) | 4.68 ± 0.06⁻³ | 8.32 ± 0.44⁻⁴ | 5.59 ± 0.42 | 3.91 ± 0.18^{-2,-4} |
| RMF_{R} | 0.130 ± 0.004⁻⁴ | 0.046 ± 0.005⁻⁵ | 0.172 ± 0.008^{-3,-6} | 0.328 ± 0.009^{-5,-7,-2} |
| (dAs/DHr) | | | | |
| AMF_{Max} | 0.386 ± 0.006⁻³ | 0.212 ± 0.003⁻⁷ | 0.347 ± 0.008^{-2,-3} | 0.552 ± 0.011^{-6,-8,-4} |
| NP | 1.74 ± 0.05⁻⁴ | 0.47 ± 0.04⁻⁶ | 1.67 ± 0.17⁻² | 4.12 ± 0.22^{-4,-6} |
| (RMF_{R}/T_{I}) × 100 | | | | |

As can be seen with reference to the Figures and Tables, simple ACP when seeded at 60 μl/ml of normal, Pi-containing SCL, induced mineral formation (T_{I}) after 6.80±0.34 h incubation. The mean rate of mineral formation during the rapid formation period (RMF_{R}) was 0.081±0.003 dAs/h; the absorbance at the ultimate amount of mineral formed (AMF_{Max.}) was projected to be 0.279±0.019 at 340 nm. The nucleation potential, (RMF_{R}/T_{I})×100, was 0.96±0.08.

Seeding with the ACP-AnxA5 complex led to quicker induction, but not more rapid or overall greater mineral formation than ACP alone. However, the nucleation potential was significantly higher. Thus, incorporation of AnxA5 during preparation of ACP had a relatively modest stimulatory effect on its ability to form mineral.

Adding type II collagen to SCL into which simple ACP was seeded caused significantly earlier induction of mineral formation (shorter T_{I}), as well as a more rapid formation rate, and a larger total amount of mineral formed than when incubated in SCL alone. The nucleation potential was also significantly higher. This shows that type II collagen enhances the rate of mineral formation when ACP is used as a nucleator, but the effects are not large (about 30-40%). Adding type II collagen to SCL into which ACP-AnxA5 was seeded had no significant effect on the T_{I}, but increased the rate and total amount of mineral formation by ˜25% when compared to incubation in SCL alone. The nucleation potential was only slightly increased. Thus addition of type II collagen to SCL had little effect on mineral formation by the ACP-AnxA5 complex.

Seeding PS-CPLX into SCL induced mineral formation more slowly than did simple ACP; once induced, its rate was less rapid, and it did not produce as much mineral as did ACP (FIG. 2A, Table IA). Its nucleation potential was also significantly lower. Thus, incorporation of PS stabilized ICP-based ACP and significantly reduced its nucleational activity. Adding type II collagen to SCL had no significant effect on T_{I}, RMF_{R} or AMF_{Max} by PS-CPLX nucleator, when compared to incubation in SCL alone. The nucleation potential also was not significantly increased. Thus, type II collagen did not stimulate mineral formation by simple PS-CPLX complex.

Seeding the ternary PS-AnxA5-CPLX complex into SCL led to 2.5-fold quicker, as well as 3.9-fold faster and 2.6-fold greater overall mineral formation, when compared to PS-CPLX alone. In addition, the nucleation potential was 9.6-fold higher. Thus, addition of AnxA5 during preparation of PS-CPLX transformed it from being a weak nucleator to one with high ability to induce and sustain mineral formation. Since in the absence of AnxA5, PS markedly inhibits mineral formation by ACP, it is apparent that AnxA5 synergistically activates PS-CPLX by forming the ternary PS-CPLX-AnxA5 complex. Adding type II collagen to SCL further enhanced mineralization of the ternary PS-CPLX-AnxA5 complex only modestly. It did not shorten the induction time, and only increased the rate, amount, and nucleation potential by only ˜15%.

While a system described herein enables accurate quantitative analysis of various features of mineral formation, it should be understood that, being a closed, controlled system, the total amount of Ca²⁺ and Pi present in each well is fixed and does not change during the course of the experiment. Thus, as mineralization begins and the amount of mineral increases, the amount of solution-phase Ca²⁺ and Pi in the SCL decreases in direct proportion. The final amount formed is dictated by the solubility product (Kₛₚ) of the solid phase, e.g. hydroxyapatite—as modified by the adsorption of components present in the system. As more solid phase forms, the driving force for its formation progressively decreases—i.e. the activity of Ca²⁺ and Pi in the solution phase decreases. The amount of mineral formed therefore reaches an asymptotic maximum that depends on the availability of ions, as well as the presence of surface-adsorbed entities that influence its Ksp. Thus, the finding that AnxA5 shortens the time of onset, as well as markedly increasing the rate and final amount of mineral formed by PS-CPLX, indicates that it not only enhances nucleation of mineral formation, but also protects the growing crystals from the adsorption of inhibitors, enabling more extensive crystal growth with improved lattice formation.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure which is herein defined and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

What is claimed is:

1. A calcium phosphate mineralization method comprising:
forming a synthetic mineralization complex, the synthetic mineralization complex including amorphous calcium phosphate and a lipid;
locating the synthetic mineralization complex in a controlled environment, the controlled environment mimicking a natural environment in which biomineralization occurs; and
monitoring the turbidity of the controlled environment according to an optical analysis technique.

2. The method according to claim 1, wherein the controlled environment is an in vitro environment.

3. The method according to claim 2, wherein the in vitro environment mimics the extracellular environment of a growth plate chondrocyte.

4. The method according to claim 2, wherein the in vitro environment mimics natural cartilage fluid.

5. The method according to claim 1, wherein the lipid is a phospholipid.

6. The method according to claim 1, wherein the synthetic mineralization complex is formed in a synthetic intracellular phosphate buffer.

7. The method according to claim 6, wherein the synthetic intracellular phosphate buffer mimics the intracellular environment of a growth plate chondrocyte.

8. The method according to claim 1, wherein the optical analysis technique comprises measuring the optical absorbency of the controlled environment.

9. The method according to claim 1, wherein the lipid is phosphatidylserine.

10. The method according to claim 1, the synthetic mineralization complex further comprising an annexin protein.

11. The method according to claim 10, wherein the annexin protein is a purified native annexin protein or a recombinant annexin protein.

12. The method according to claim 1, the controlled environment further comprising collagen.

13. A method for examining the kinetics of the formation of a biomimetic solid phase comprising:
forming a biomimetic solid phase in an environment;
monitoring the turbidity of the environment according to a non-radioactive optical analysis technique, wherein the optical analysis technique does not physically disturb the environment;
gathering the turbidity data over a period of time; and
carrying out a first derivative analysis of the gathered data to determine a kinetic parameter of the formation of the biomimetic solid phase.

14. The method according to claim 13, wherein the biomimetic solid phase comprises calcium phosphate mineral.

15. The method according to claim 13, wherein the biomimetic solid phase is a biofilm.

16. The method according to claim 13, wherein the step of monitoring the turbidity of the environment comprises measuring the optical absorbency of the environment.

17. The method according to claim 16, wherein the turbidity data over time describes a quasi-sigmoidal pattern represented by the equation:
$$y = d + \frac{( a - d )}{{( 1 + {( \frac{x}{c} )}^{b} )}^{g}}$$
wherein x is absorbency and
y is time,
the method further comprising solving the equation for a, b, c, d, and g.

18. A system for examining a mineralization process comprising:
a controlled environment for containing a biomimetic mineral deposition, the controlled environment including a synthetic mineralization complex, the synthetic mineralization complex including amorphous calcium phosphate and a lipid; and
an optical device in optical communication with the controlled environment, wherein the optical device monitors the turbidity of the controlled environment.

19. The system according to claim 18, wherein the controlled environment in an in vitro environment.

20. The system according to claim 18, wherein the lipid is a phospholipid.

21. The system according to claim 20, wherein the phospholipid is phosphatidylserine.

22. The system according to claim 18, the synthetic mineralization complex further including an annexin protein.

23. The system according to claim 22, wherein the annexin protein a purified native annexin protein or a recombinant annexin protein.

24. The system according to claim 18, wherein the controlled environment mimics a natural extracellular environment.

25. The system according to claim 24, wherein the natural extracellular environment is the extracellular environment of a growth plate chondrocyte.

26. The system according to claim 24, wherein the natural extracellular environment mimics cartilage lymph, blood, or serum.

27. The system according to claim 18, the controlled environment further comprising collagen.

28. The system according to claim 18, wherein the optical device monitors the turbidity of the controlled environment by measuring the absorbency of the controlled environment.

29. The system according to claim 18, further comprising a data analysis component in communication with the optical device for receiving turbidity data from the optical device.

30. The system according to claim 29, the data analysis component comprising software for mathematical manipulation of turbidity data obtained from the optical device.
